(19) United States
(12) Patent Application Publication
(10) Pub. No.: US 2022/0333109 A1
(43) Pub. Date: Oct. 20, 2022

(54) Long Noncoding RNA Implicated in Cardiovascular Disease and Use Thereof

(71) Applicant: INDUSTRY-ACADEMIC COOPERATION FOUNDATION, YONSEI UNIVERSITY, Seoul (KR)

(72) Inventors: Sang Hak Lee, Seoul (KR); Soo-Jin Ann, Seoul (KR)

(21) Appl. No.: 17/715,842

(22) Filed: Apr. 7, 2022

(30) Foreign Application Priority Data
Apr. 8, 2021 (KR) 10-2021-0045769

(51) Int. Cl. C12N 15/113; C12N 15/11; A61K 31/713; A61K 47/54; C12Q 1/68

(52) U.S. Cl. CPC C12N 15/113; C12N 15/111; A61K 31/713; A61K 47/549; C12Q 1/68; C12N 2310/346; C12N 2310/113

(57) ABSTRACT

The present invention relates to a composition for diagnosing cardio-cerebrovascular disease and a composition for preventing or treating cardio-cerebrovascular disease. The present invention may provide important clinical information that enables early establishment of treatment strategies by highly reliable prediction of not only the development of cardio-cerebrovascular diseases, including arteriosclerosis, but also the likelihood of future development of cardio-cerebrovascular diseases, based on the expression of lncRNA HSPA7. In addition, the composition for treating cardio-cerebrovascular disease according to the present invention inhibits HSPA7 expression, thereby inhibiting the migration of smooth muscle cells and decreasing the expression of inflammatory factors to block the development and progression of atherosclerotic plaques themselves, and thus it may be effectively used as a fundamental therapeutic composition that goes beyond symptomatic therapy such as administration of antithrombotic agents.

### CROSS-REFERENCE TO RELATED APPLICATIONS

This application claims the benefit of priority of Korean Patent Application no. 10-2021-0045769, filed Apr. 8, 2021, which is hereby incorporated herein by reference in its entirety.

### SEQUENCE LISTING STATEMENT

This application includes an electronically submitted Sequence Listing in text format. The text file contains a sequence listing entitled “ 22-0617-US_ST25.txt” created on Apr. 7, 2022 and is 69 kilobytes in size. The Sequence Listing contained in this text file is part of the specification and is hereby incorporated by reference herein in its entirety.

### BACKGROUND

### 1. Technical Field

The present invention relates to a method of diagnosing cardiovascular disease using, as a biomarker, lncRNA HSPA7 newly found to be involved in cardiovascular disease, and a method of preventing or treating cardiovascular disease by inhibiting the expression of lncRNA HSPA7.

### 2. Related Art

The risk of atherosclerotic cardiovascular diseases, such as coronary artery disease, is strongly attributable to genetic factors (Lloyd-Jones, JAMA 2004). According to recent genetic studies, many novel genetic loci are associated with coronary artery disease but have an unknown function, and many of them are reportedly located in noncoding regions of the human genome (McPherson, Circ Res 2016). For example, the association between the 9p21 locus and myocardial infarction has been replicated by research groups, and ANRIL, a noncoding RNA, was found to be located in this locus and to influence cell proliferation (Congrains, Atherosclerosis 2012).

Long noncoding RNAs (lncRNAs) are the longest types of noncoding RNAs and are differentiated from other shorter noncoding RNAs. lncRNAs are known to have very diverse functions and are regarded as attractive therapeutic targets due to their tissue, cell, and disease specificities (Pierce, ATVB 2020). As lncRNAs affect distal targets, they stabilize ribonucleoprotein complexes, alter phosphorylation pathways, or act as competing endogenous RNAs (Fasolo, Cariovasc Res 2019). Dozens of lncRNAs have been reported to affect the development of atherosclerosis. Prior studies showed that specific lncRNAs regulate cells in arteries, including endothelial cells, vascular smooth muscle cells (VSMCs), and macrophages (Pierce, ATVB 2020). For instance, lncRNA H-19 increases VSMC proliferation (Ly, BBRC 2018), whereas lncRNA-p21 inhibits VSMC proliferation (Wu, Circulation 2014). Although some additional lncRNAs were identified to regulate vascular cells (Zhang, JACC 2018), lncRNAs derived from human vascular cells that affect atherosclerosis have been highly limited. Meanwhile, microRNAs (miRNAs) inhibit target mRNAs via degradation or translational blocking (Ono, FEBS J 2020). Some miRNAs are known to be involved in atherosclerosis by affecting SMC proliferation or phenotypic changes (Lu, ATVB 2018).

The present inventors have analyzed human atherosclerotic plaques to identify lncRNA having unknown function and to find their function.

Throughout the present specification, a number of publications and patent documents are referred to and cited. The disclosure of the cited publications and patent documents is incorporated herein by reference in its entirety to more clearly describe the state of the related art to which the present invention pertains and the content of the present invention.

### SUMMARY

The present inventors have made extensive efforts to discover effective biomarkers that can accurately predict the genetic risk of cardio-cerebrovascular diseases, including coronary artery disease. As a result, the present inventors have found that HSPA7, a long non-coding RNA, is specifically and highly expressed in atherosclerotic plaques, and its expression promotes migration of human aortic smooth muscle cells (HASMCs) and increases expression of inflammatory factors to induce cardio-cerebrovascular injury and blood flow disorder, thereby completing the present invention.

Therefore, an object of the present invention is to provide a composition for predicting or diagnosing cardio-cerebrovascular disease.

Another object of the present invention is to provide a composition for preventing or treating cardio-cerebrovascular disease.

Still another object of the present invention is to provide a method for screening a composition for preventing or treating cardio-cerebrovascular disease.

Other objects and advantages of the present invention will become more apparent from the following detailed description of the invention, the appended claims and the accompanying drawings.

According to one aspect of the present invention, the present invention provides a composition for predicting or diagnosing cardio-cerebrovascular disease containing, as an active ingredient, an agent for measuring the expression level of lncRNA HSPA7.

The present inventors have made extensive efforts to discover biomarkers that can accurately predict the genetic risk of cardio-cerebrovascular diseases, including coronary artery disease. As a result, the present inventors have found that HSPA7, a long non-coding RNA, is specifically and highly expressed in atherosclerotic plaques, and its expression promotes migration of human aortic smooth muscle cells (HASMCs) and increases expression of inflammatory factors to induce cardio-cerebrovascular injury and blood flow disorder. Accordingly, the present inventors have first found that HSPA7 is not only a highly reliable predictive and diagnostic marker but also an effective therapeutic target for cardio-cerebrovascular disease.

As used herein, the term “cardio-cerebrovascular disease” refers to a series of diseases that cause abnormalities in blood vessels supplying blood to the brain and heart, resulting in decreased blood flow and consequent ischemic tissue injury, and is a generic term for antecedent diseases such as ischemic heart disease, cerebrovascular disease, hypertension, diabetes, dyslipidemia, and arteriosclerosis.

Examples of cardio-cerebrovascular disease that can be prevented or treated with the composition of the present invention include, but are not limited to, myocardial infarction, atherosclerosis, atherothrombosis, coronary artery disease, stable and unstable angina, stroke, vascular stenosis, vascular restenosis, aortic aneurysms, and acute ischemic arteriovascular events.

As used herein, the term “diagnosis” or “diagnosing” refers to determining the susceptibility of a subject to a specific disease, determining whether a subject currently has a specific disease, or determining the prognosis of a subject with a specific disease.

As used herein, the term “composition for diagnosing” refers to a mixture or device including a means for measuring the expression level of HSPA7 to determine the development of epithelial barrier dysfunction or the likelihood of development of epithelial barrier dysfunction in a subject, and thus may also be expressed as a “diagnostic kit”.

According to a specific embodiment of the present invention, the agent for measuring the expression level of lncRNA HSPA7 is a primer or a probe that specifically binds to the nucleotide of SEQ ID NO: 1.

As used herein, the term “nucleotide” is meant to comprehensively include DNA (gDNA and cDNA) and RNA molecules. Nucleotides that are the basic units of the nucleic acid molecule include naturally occurring nucleotides as well as analogues with modified sugars or bases.

According to the present invention, the nucleotide sequence of SEQ ID NO: 1 is the RNA nucleotide sequence of HSPA7. It is obvious to those skilled in the art that the nucleotide sequence used that is used in the present invention is not limited to the nucleotide sequence of SEQ ID NO: 1.

Considering variations having a biological activity equivalent to that of the nucleotide sequence, it is construed that examples of the nucleotide that is used in the present invention also include sequences showing substantial identity with a naturally occurring HSPA7 sequence. The term “sequences showing substantial identity” means sequences that shows an identity of at least 70%, specifically at least 80%, more specifically at least 90%, most specifically at least 95%, as determined by aligning the sequence of the present invention to any other sequence so as to correspond to each other to the greatest possible extent and analyzing the aligned sequence using an algorithm commonly used in the art. Alignment methods for sequence comparison are known in the art. Various methods and algorithms for alignment are disclosed in Huang et al., Comp. AppL BioSci. 8:155-65(1992) and Pearson et al., Meth. Mol. Biol. 24:307-31(1994). NCBI Basic Local Alignment Search Tool (BLAST) (Altschul et al., J. Mol. Biol. 215:403-10 (1990)) is accessible from the National Center for Biological Information (NBCI) and the like and may be used in conjunction with sequence analysis programs such as blastp, blasm, blastx, tblastn and tblastx on the Internet.

As used herein, the term “primer” refers to an oligonucleotide which acts as a point of initiation of synthesis under conditions in which synthesis of a primer extension product complementary to a nucleic acid strand (template), that is, conditions including the presence of nucleotides and a polymerase such as a DNA polymerase, and a suitable temperature and pH. Specifically, the primer is a single-stranded deoxyribonucleotide. Examples of the primer used that is in the present invention include naturally occurring dNMPs (i.e., dAMP, dGMP, dCMP and dTMP), modified nucleotides, or non-natural nucleotides, as well as ribonucleotides.

The primer of the present invention may be an extension primer that is annealed to the target nucleic acid to form a sequence complementary to the target nucleic acid by a template-dependent nucleic acid polymerase, and the extension primer extends to a position where the immobilization probe is annealed and occupies the area where the probe is annealed.

The extension primer that is used in the present invention includes a hybridizing nucleotide sequence complementary to a specific nucleotide sequence of a target nucleic acid, for example, HSPA7. As used herein, the term “complementary” means that a primer or probe is sufficiently complementary to a target nucleic acid sequence so as to hybridize selectively to the target nucleic acid sequence under certain annealing or hybridization conditions, and is meant to include both substantially complementary and perfectly complementary, and specifically refers to perfectly complementary. As used herein, the term “substantially complementary sequence” is meant to include not only a perfectly matched sequence, but also a sequence that is partially mismatched with the sequence to be compared, within the range in which the sequence can function as a primer by annealing to a specific sequence.

The primer should be long enough to prime the synthesis of extension products in the presence of a polymerase. A suitable length of the primer is determined depending on a number of factors, such as temperature, pH, and the source of the primer, but is typically 15 to 30 nucleotides. Short primer molecules generally require lower temperatures to form sufficiently stable hybrid complexes with the template. The design of such a primer can be easily performed by those skilled in the art with reference to the target nucleotide sequence, for example, using a primer design program (e.g., PRIMER 3 program).

As used herein, the term “probe” refers to natural or modified monomers, including deoxyribonucleotides and ribonucleotides capable of hybridizing to a specific nucleotide sequence, or linear oligomers having linkages. Specifically, the probe is single-stranded for maximum efficiency in hybridization, and is more specifically a deoxyribonucleotide. As the probe that is used in the present invention, a sequence perfectly complementary to the specific nucleotide sequence of HSPA7 may be used, but a substantially complementary sequence may be used within a range in which the sequence does not interfere with specific hybridization. In general, since the stability of a duplex formed by hybridization generally tends to be determined by the match of the sequences at the ends, a probe complementary to the 3′-end or 5′-end of the target sequence is preferably used.

Suitable conditions for hybridization may be determined by referring to Joseph Sambrook, et al., Molecular Cloning, A Laboratory Manual, Cold Spring Harbor Laboratory Press, N.Y. (2001) and Haymes, B. D., et al., Nucleic Acid Hybridization, A Practical Approach, IRL Press, Washington, D.C. (1985).

According to another aspect of the present invention, the present invention provides a composition for preventing or treating cardio-cerebrovascular disease containing an inhibitor for lncRNA HSPA7 expression as an active ingredient.

Since cardio-cerebrovascular disease to be diagnosed, predicted, treated or prevented in the present invention has already been described above, description thereof will be omitted to avoid excessive complexity of the present specification.

As used herein, the terms “inhibitor for expression” refers to a substance that decreases the expression or activity of HSPA7. Specifically, the term means a substance that decreases the expression or activity of HSPA7, thereby making the expression or activity of HSPA7 undetectable or insignificant and significantly ameliorating atherosclerotic plaques or vascular injury caused by HSPA7.

As used herein, the term “decreased expression” refers to a state in which the expression level of HSPA7 has decreased by, for example, at least 20%, more specifically at least 30%, even more specifically at least 40%, compared to a control.

As used herein, the term “treatment” or “treating” refers to: (a) inhibiting the development of a disease, disorder or symptom; (b) alleviating the disease, disease or symptom; or (c) eliminating the disease, disease or symptom. When the composition of the present invention is administered to a subject, it acts to inhibit HSPA7 expression, thereby inhibiting the migration of smooth muscle cells and decreasing the expression of inflammatory factors to block or delay the development and progression of atherosclerotic plaques, thereby suppressing the development of or eliminating or alleviating symptoms caused by various cardio-cerebrovascular diseases, including arteriosclerosis. Thus, the composition of the present invention may be used alone to treating these diseases, or may be applied as a therapeutic adjuvant for the above-described diseases by being administered together with other pharmacological components. Accordingly, as used herein, the term “treatment” or “therapeutic agent” includes the meaning of “adjuvant treatment” or “therapeutic adjuvant”.

As used herein, the term “administration” or “administering” refers to administering a therapeutically effective amount of the composition of the present invention directly to a subject so that the same amount is formed in the body of the subject.

As used herein, the term “therapeutically effective amount” refers to the content of the pharmacological ingredient in the pharmaceutical composition, which is sufficient to provide a therapeutic or prophylactic effect to a subject to whom the pharmaceutical composition of the present invention is to be administered. Thus, the term is meant to include “a prophylactically effective amount”.

As used herein, the term “subject” includes, without limitation, humans, mice, rats, guinea pigs, dogs, cats, horses, cows, pigs, monkeys, chimpanzees, baboons, or rhesus monkeys. Specifically, the subject in the present invention is a human.

According to a specific embodiment of the present invention, examples of the lncRNA HSPA7 expression inhibitor include, but are not limited to, shRNA, siRNA, miRNA, ribozyme, PNA, and antisense oligonucleotides, which bind specifically to the nucleotide sequence of SEQ ID NO: 1. In addition, as the lncRNA HSPA7 expression inhibitor, any nucleic acid molecule including a complementary nucleic acid sequence capable of hybridizing to lncRNA HSPA7 may be used which is capable of specifically recognizing HSPA7 and causing a modification in the nucleotide structure, which causes deterioration of the function of HSPA7.

As used herein, the term “small hairpin RNA (shRNA)” refers to a single-stranded RNA sequence consisting of 50 to 70 nucleotides, which forms a stem-loop structure in vivo and creates a tight hairpin structure for inhibiting target gene expression by RNA interference. Usually, a double-stranded stem is formed by complementary base pairing of a long RNA consisting of 19 to 29 nucleotides on both sides of the loop consisting of 5 to 10 nucleotides, and for constitutive expression, the double-stranded stem is transduced into cells via a vector including U6 promoter and is usually delivered to daughter cells so that inhibition of expression of the target gene is inherited.

As used herein, the term “siRNA” refers to a short double-stranded RNA capable of inducing RNA interference (RNAi) through cleavage of a specific RNA. The siRNA is composed of a sense RNA strand having a sequence homologous to the RNA of the target gene and an antisense RNA strand having a sequence complementary thereto. The total length thereof may be 10 to 100 bases, preferably 15 to 80 bases, most preferably 20 to 70 bases, and the ends thereof may be blunt or cohesive as long as the siRNA is capable of inhibiting the expression of the target gene by the RNAi effect. The cohesive ends may have a 3′ end overhang and a 5′ end overhang.

As used herein, the term “microRNA (miRNA)” refers to an oligonucleotide that is not expressed in cells, and means a single-stranded RNA molecule that inhibits target gene expression by complementary binding to the target RNA while having a short stem-loop structure.

As used herein, the term “ribozyme” refers to a kind of RNA which is an RNA molecule having the same function as an enzyme that recognizes and cleaves a specific RNA nucleotide sequence by itself. The ribozyme is a nucleotide sequence complementary to a target RNA strand and is composed of a region that binds specifically to the target RNA and a region that cleaves the target RNA.

As used herein, the term “peptide nucleic acid (PNA)” refers to a molecule capable of complementary binding to DNA or RNA while having both nucleic acid and protein properties. PNA is not found in nature, is artificially synthesized by a chemical method, and regulates target gene expression by forming a double strand through hybridization with a natural nucleic acid having a nucleotide sequence complementary thereto.

As used herein, the term “antisense oligonucleotide” refers to a nucleotide sequence complementary to a specific RNA sequence. Specifically, it refers to a nucleic acid molecule which binds to a complementary sequence in a target RNA and inhibits the activities of the target RNA, which are essential for translation into proteins, translocation into cytoplasm, maturation, or all other overall biological functions. The antisense oligonucleotide may be modified at one or more bases, sugars or backbone positions to enhance efficacy thereof (De Mesmaeker et al., Curr Opin Struct Biol., 5(3):343-55, 1995). The oligonucleotide backbone may be modified with phosphorothioate, phosphotriester, methylphosphonate, short-chain alkyl, cycloalkyl or short-chain heteroatomic linkages.

The above-described nucleic acid molecule for inhibiting expression according to the present invention may be expressed in a subject with cardio-cerebrovascular disease, thereby inhibiting the expression of lncRNAHSPA7.

As used herein, the term “expressing” or “expression” means allowing a subject to express an exogenous gene or artificially introducing an endogenous gene using a gene delivery system to increase the natural expression level of the endogenous gene, thereby making the introduced gene replicable as an extrachromosomal factor or by chromosomal integration in a cell. Accordingly, the term “expression” is synonymous with “transformation”, “transfection” or “transduction”.

As used herein, the term “gene delivery system” refers to any means for delivering a gene into a cell, and the term “gene delivery” has the same meaning as intracellular transduction of a gene.

At the tissue level, the term “gene delivery” has the same meaning as the spread of a gene. Accordingly, the gene delivery system of the present invention may be referred to as a gene transduction system or a gene spread system.

According to a specific embodiment of the present invention, the composition of the present invention inhibits the migration of smooth muscle cells.

According to a specific embodiment of the present invention, the composition of the present invention reduces the secretion of interleukin (IL)-1β and IL-6.

According to another aspect of the present invention, the present invention provides a method for screening a composition for preventing or treating cardio-cerebrovascular disease, the method comprising steps of:

(a) bringing a candidate substance into contact with a biological sample containing lncRNA HSPA 7-expressing cells; and (b) measuring the expression level of lncRNA HSPA7 in the sample, wherein the candidate substance is determined as the composition for preventing or treating cardio-cerebrovascular disease, when the expression level of lncRNA HSPA7 has decreased.

As used herein, the term “biological sample” refers to any sample containing lncRNA HSPA 7-expressing cells, obtained from mammals including humans. Examples of the biological sample include, but are not limited to, tissues, organs, cells, or cell cultures.

According to a specific embodiment of the present invention, the lncRNA HSPA7-expressing cells are vascular cells, and more specifically, vascular smooth muscle cells.

The term “candidate substance” as used while referring to the screening method of the present invention refers to an unknown substance that is added to the sample containing HSPA 7-expressing cells and is used in screening to test whether it affects the activity or expression level of HSPA7. Examples of the candidate substance include, but are not limited to, compounds, nucleotides, peptides, and natural extracts. The step of measuring the activity or expression level of HSPA7 in the biological sample treated with the candidate substance may be performed by various measurement methods known in the art, and when the activity or expression level of HSPA7 has decreased as a result of the measurement, the candidate substance may be determined as the composition for preventing or treating cardio-cerebrovascular disease.

As used herein, the term “decreased activity or expression level” means that smooth muscle cell migration and inflammatory factor expression induced by lncRNA HSPA7 are significantly inhibited, so that the expression level or in vivo intrinsic function of lncRNA HSPA7 is reduced such that atherosclerotic plaque generation and vascular injury are decreased to measurable levels. Decreased activity includes not only a simple decrease in function, but also eventual inhibition of activity due to decreased stability. Specifically, it may refer to a state in which the activity or expression level has decreased by at least 20%, more specifically at least 40%, even more specifically at least 60%, compared to a control.

### BRIEF DESCRIPTION OF THE DRAWINGS

FIGS. 1A and 1B, together with Table 1, show that HSPA7 expression is upregulated in human atherosclerotic plaques and induced by atherogenic stimuli. FIG. 1A is a Heatmap of genes differentially expressed in human atherosclerotic plaques compared to control tissues. Table lshows a list of high-ranking lncRNAs with up- or downregulated expression in plaques. FIG. 1B shows the results of performing hierarchical clustering and multidimensional scaling of data from plaques and controls. FIG. 1C shows the results of verifying differently expressed lncRNA through RNA-sequencing. Among the three top-ranked genes, only HSPA7 showed a significant elevation of expression upon proatherogenic stimuli, particularly oxLDL. Experiments were conducted in duplicates, and data were obtained from three independent experiments.

FIGS. 2A-2E show that knockdown of HSPA7 attenuates migration and inflammatory changes in HASMC. FIGS. 2A and 2B show the results of Transwell assay performed to assess cell migration of HASMCs transfected with siHSPA7 or control siRNA for 24 hours. FIG. 2C shows the results of ELISA and qPCR analyses performed to examine the secretion patterns of IL-1β and IL-6 upon treatment with oxLDL and siHSPA7. FIG. 2D shows the results of immunofluorescence staining performed to examine the expression patterns of CD68, SM22α and calponin1 upon treatment with oxLDL and siHSPA7. FIG. 2E shows the result of qPCR performed to evaluate the effect of HSPA7 on phenotypic markers, particularly CNN1 and CD68. Data were obtained from three independent experiments.

FIGS. 3A-3F show that HSPA7 promotes inflammatory changes in HASMCs by sponging miR-223. FIG. 3A shows the results of bioinformatics analysis using miRcode, which predicted that the HSPA7 sequence contains a potential miR-223 binding site. FIG. 3B shows qPCR results indicating that FOXO1 expression was upregulated in HASMCs treated with a miR-223 inhibitor, but diminished after siHSPA7 treatment. NF-κB activity was elevated upon miR-223 inhibition, but attenuated after siHSPA7 treatment. FIG. 3C shows that, after the same treatment of HASMCs, the secretion of IL-1β and IL-6 was increased by a miR-223 inhibitor, but decreased after siHSPA7 treatment, as verified by qPCR. FIG. 3D shows immunofluorescence staining results indicating that the expression of markers of the contractile SMC phenotype was not changed by a miR-223 inhibitor, but was increased after siHSPA7 addition. FIG. 3E shows the results of verifying the above results using qPCR for TAGLN and CNN1. FIG. 3F shows that miR-223 is a target of HSPA7 in an AGO2-dependent manner. AGO2 expression was not different in HASMCs regardless of the presence of oxLDL. RIP assays showed that HSPA7 and miR-223 were more enriched in AGO2-containing miRNPs than IgG immunoprecipitates. HSPA7 had increased binding to AGO2 in the presence of oxLDL. Data were obtained from three independent experiments.

*: p<0.05, AngII: Angiotensin II, HASMC: human aortic smooth muscle cell; oxLDL: oxidized low-density lipoprotein; C: control; AGO: argonaute-2; RIP: RNA immunoprecipitation; miRNP: miRNA ribonucleoprotein; ELISA: enzyme-linked immunosorbent assay; qPCR: quantitative real-time polymerase chain reaction.

### DETAILED DESCRIPTION

Hereinafter, the present invention will be described in more detail with reference to examples. These examples are only for illustrating the present invention in more detail, and it will be obvious to those skilled in the art that the scope of the present invention according to the subject matter of the present invention is not limited by these examples.

### EXAMPLES

### Experimental Methods

### Study Subjects and Aortic Tissue Extraction

The study protocol (no. 4-2013-0688) was approved by the Institutional Review Board of Severance Hospital, Seoul, Korea. All participants provided written informed consent. Aortic samples were obtained from patients who underwent aortic graft replacement surgery for a thoracic aortic aneurysm. Samples were classified by an experienced pathologist according to the presence of atherosclerotic plaques. The lesions were classified according to the modified classification of the American Heart Association (Virmani, ATVB 2000) without any knowledge of the specimens.

### RNA Sequencing and Classification of lncRNAs

RNA was extracted from cells using a Ribospin RNA Extraction Kit (GeneAll, Seoul, Korea). RNA concentration and purity were assessed using a NanoDrop ND1000 spectrophotometer. Total RNA sequencing libraries were prepared using a TruSeq RNA sample preparation kit (Illumina, San Diego, Calif., USA) according to the manufacture's instruction. The differentially expressed genes between samples with and without atherosclerotic plaques were compared using Cuffdiff. Genes with q values <0.05 and >two-fold changes were identified.

### Human Aortic Smooth Muscle Cells (HASMCs) and Other Reagents

HASMCs were purchased from Lonza (Basel, Switzerland) and cultured in SMC basal growth medium (containing growth factor and supplemented with 2% fetal bovine serum and penicillin/streptomycin) at 37° C. Lipopolysaccharide (LPS) and angiotensin II (AngII) were purchased from Sigma-Aldrich (St. Louis, Mo., USA) and used for HASMC stimulation. Low-density lipoprotein (LDL) was isolated from the plasma of healthy donors using sequential ultracentrifugation. LDL was dialyzed for 24 hours at 4° C. with phosphate-buffered saline and oxidized for 24 hours using 5 μM CuSO₄ at 37° C. Ethylenediaminetetraacetic acid was added to stop the reaction, and thiobarbituric acid reactive substance (TBARS) assays were used to analyze the oxidation state of LDL before each experiment.

### Migration Assay

For analysis and comparison of HASMC migration, the cells were added to the upper Transwell chamber (Neuro Probe, Inc., Gaithersburg, Md., USA) in serum-free medium after transfection with siHSP A7 or control siRNA for 24 hours. The lower chamber was filled with SMC growth basal medium with fetal bovine serum. Next, LPS (10 ng/mL), oxLDL (50 μg/mL), or AngII (300 nM) was added to the upper chamber and incubated for another 24 hours. Thereafter, the cells were stained with a Diff Quik staining kit (Kobe, Japan), and those on the lower surface of the filter were photographed and counted under a fluorescence microscope. All treatments were performed in duplicate wells.

### RNA Sequencing and Analysis of lncRNAs

The fragmentation step resulted in an RNA-Seq library that included inserts of approximately 100 to 400 bp. The average insert size in an Illumina TruSeq library was approximately 200 bp. cDNA fragments underwent an end repair process: the addition of a single ‘A’ base to the 3′ end and then ligation of adapters. Finally, the products were purified and enriched with polymerase chain reaction (PCR) to obtain double-stranded cDNA libraries. Libraries were quantified using KAPA Library Quantification kits for the Illumina HiSeq 2500 platform according to the protocol guide KK4855 (KAPA Biosystems, Wilmington, Mass., USA).

### Enzyme-Linked Immunosorbent Assay (ELISA)

Cell culture supernatants were collected, and the amount of IL-1β or IL-6 was quantified using an ELISA kit (R&D Systems, Minneapolis, Minn., USA) according to the manufacturer's protocol. 96-well plates were coated with 1 mg/well capture antibody. The coated plates were washed twice with phosphate-buffered saline containing 0.05% Tween-20 and were exposed to biotin-conjugated secondary antibodies. The plates were read at an absorbance of 450 nm. Target proteins were analyzed according to the manufacturer's protocol.

### Immunoblot Assay

Cells were harvested and lysed in cell lysis buffer containing 1M HEPES (pH 7.5), 5M NaCl, 0.5M EDTA, 1% Triton X-100 and protease inhibitor cocktail (Roche, Basel, Switzerland). Protein concentration was measured by bicinchoninic acid protein assay (Pierce Biotechnology Inc., Waltham, Mass., USA). Cell lysates (20 mg/lane) were subjected to 12.5% sodium dodecyl sulfate-polyacrylamide gel electrophoresis and transferred to polyvinylidene difluoride membranes (Merck Millipore, Burlington, Mass., USA). The membranes were incubated with 5% bovine serum albumin in Tween 20-containing TBS (Tris-buffered saline) solution for 1 hour at room temperature and incubated with primary antibody overnight at 4° C. The next day, the membranes were washed with TBS and incubated with horseradish peroxidase-conjugated secondary antibody at room temperature. Proteins were detected with an enhanced chemiluminescence system.

### Quantitative Real-Time PCR (RT-qPCR)

RNA was extracted from cells using a Ribospin RNA Extraction Kit (GeneAll, Seoul, Korea). The integrity of the extracted RNA was analyzed with a NanoDrop and quantified using spectrophotometric absorbance at 260 nm. Next, 1 μg of RNA was synthesized into cDNA using an iScript™ cDNA Synthesis kit (Bio-Rad, Hercules, Calif., USA). RT-qPCR was performed with a SYBR Green dye system on a LightCycler 480 real-time PCR machine (Roche, Basel, Switzerland). LightCycler software was used to analyze gene expression based on cycle threshold values normalized to β-actin expression. Amplified gene expression was assessed by melting curve analysis, and no reverse transcriptase or template controls were included. Analyses were performed in duplicates.

### RNA-Binding Protein Immunoprecipitation (RIP) Assay

The RIP assay was conducted using an EZ-Magna RIP kit (Merck Millipore, Burlington, Mass., USA) according to the manufacturer's instructions. HASMCs were harvested and lysed in RIP lysis buffer. Cell extracts were then incubated with RIP buffer containing magnetic beads conjugated with anti-AGO2 antibody and IgG (Merck Millipore). Immunoprecipitated RNA was isolated, and qPCR analysis was performed to detect HSPA7 and miR-223.

### Statistical Analysis

All data are presented as the mean ±standard error. Analysis of variance, followed by Tukey's test, was used to compare values of continuous variables between groups with post hoc analysis. Differences were considered statistically significant when the p value was p<0.05. The software package Prism 5.0 was used for all data analyses (GraphPad Software Inc., San Diego, Calif., USA).

### Experimental Results

### Identification of lncRNAs Associated With Human Atherosclerosis

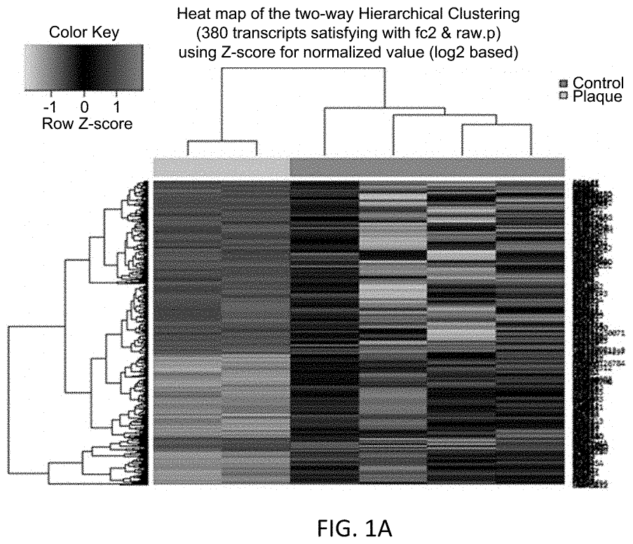

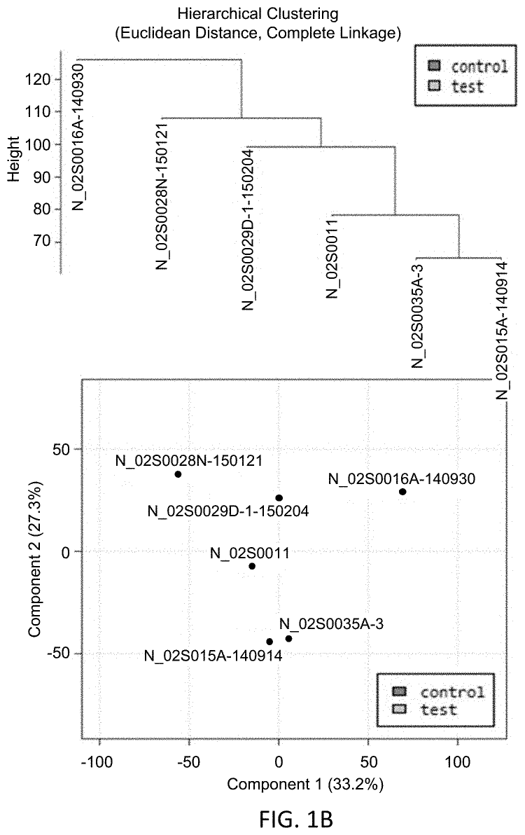

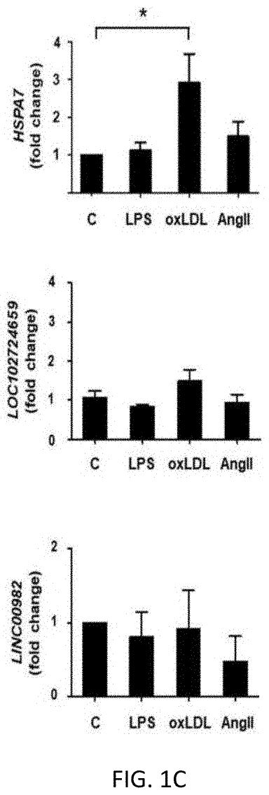

RNA sequencing was performed to identify lncRNAs associated with atherosclerotic plaques. A total of 380 RNAs were found to be differentially expressed between plaques and controls (FIG. 1A). Hierarchical clustering and multidimensional scaling were conducted based on fragments per kilobase of transcripts per million mapped reads values (FPKM) (fold change >2; p<0.05), and the gene expression pattern in plaques was distinct from that of the controls. Table 1 shows high-ranking lncRNAs with up- or downregulated expression in plaques.

TABLE 1

| | IncRNA | | | | Fold | |
|---|---|---|---|---|---|---|
| | accession | Chromosome | Start | Stop | change | ρ |
| Up- | HSPA7 | 1 | 161606291 | 161608217 | 6.13 | 4.50E−02 |
| regulated | TYROBP | 19 | 35904401 | 35908309 | 2.85 | 2.70E−02 |
| | LIPE-AS1 | 19 | 42397148 | 42652355 | 2.79 | 1.50E−03 |
| | PRDM16 | 1 | 3059617 | 3067725 | 2.36 | 2.30E−02 |
| | LOC102724659 | 3 | 46364660 | 46407059 | 2.18 | 4.64E−03 |
| | LAIR1 | 19 | 54353624 | 54370556 | 2.09 | 2.11E−02 |
| | SLC7A7 | 14 | 22773222 | 22619811 | 2.04 | 3.91E−04 |
| Down- | MIR4697HG | 11 | 133696435 | 133901740 | 2.07 | 1.88E−03 |
| regulated | LINC00982 | 1 | 3059617 | 3067725 | 2.37 | 1.00E−03 |
| | LINC00312 | 3 | 8571782 | 8574688 | 2.40 | 1.44E−02 |
| | NAV2-A56 | 11 | 19710934 | 19714672 | 2.86 | 1.00E−03 |

Multidimensional scaling visualized differences in gene expression between the groups (FIG. 1B). Three lncRNAs (HSPA7, LOC102724659, and LINC00982) showing significantly different expression were selected and subjected to additional experiments. When HASMCs were incubated with LPS, oxLDL, or AngII, only HSPA7 showed upregulated expression, whereas the expression of the other two lncRNAs was not substantially changed (FIG. 1C).

### Knockdown of HSPA7 Attenuates Migration and Inflammatory Changes in HASMCs

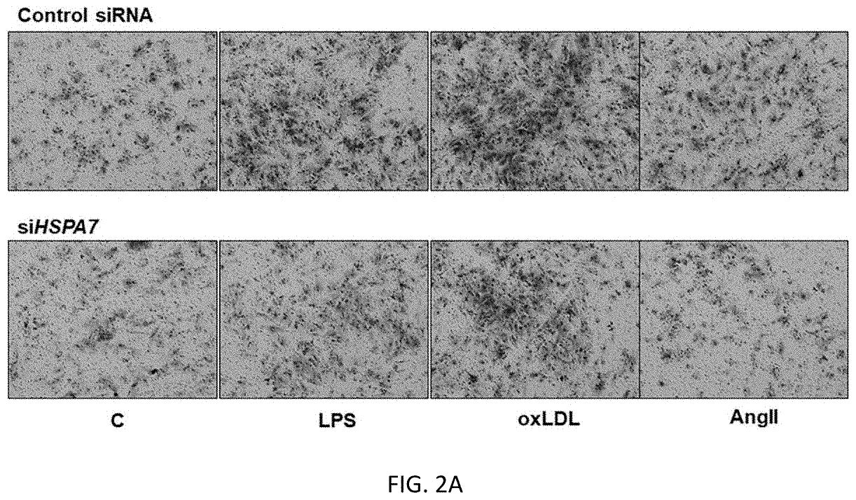

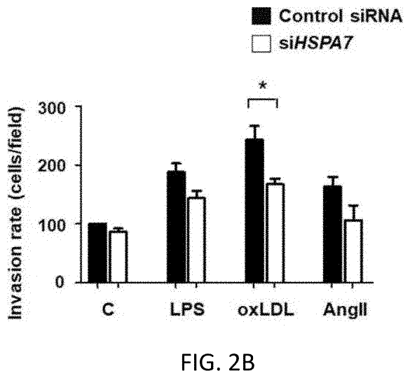

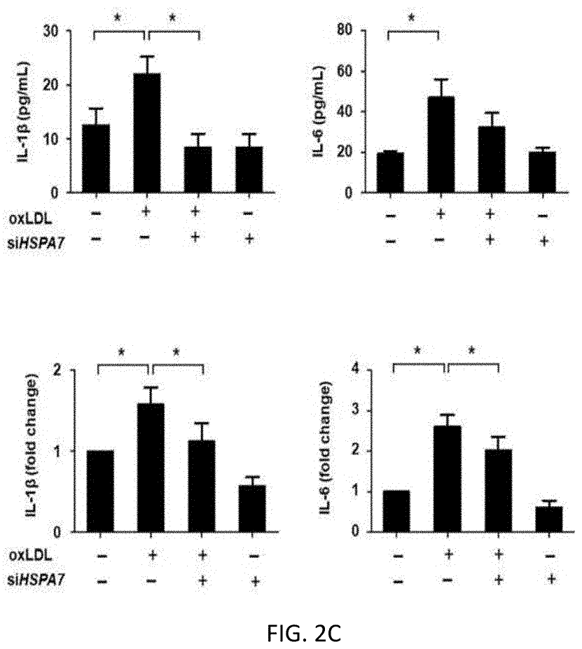

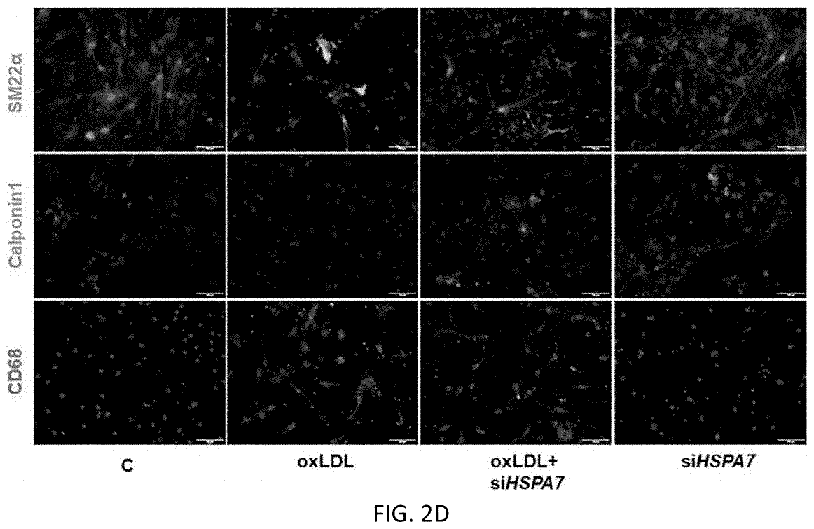

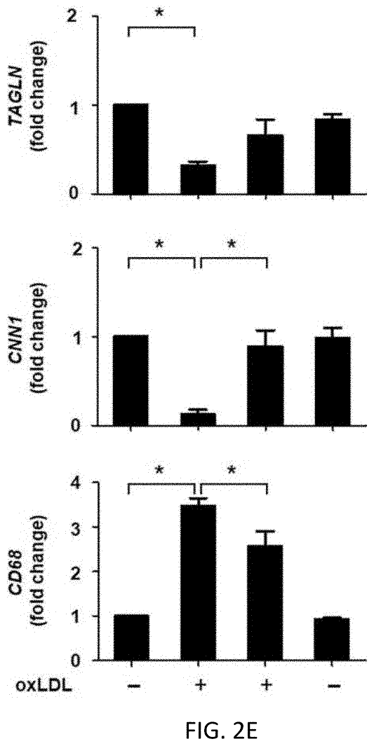

To examine the effect of HSPA7 knockdown on HASMC migration, the cells were transfected with siHSPA7 or control siRNA for 24 hours and plated in the upper Transwell chamber with or without LPS, oxLDL, or AngII. After another 24 hours, analysis of the cells in the lower chamber revealed that migration of HASMCs promoted by oxLDL was significantly inhibited after siHSPA7 treatment (FIGS. 2A and 2B). After transfection with siHSPA7 or control siRNA, HASMCs were treated with or without oxLDL for 24 hours. ELISAs and qPCR showed that the oxLDL-promoted secretion and expression of IL-1β and IL6 were suppressed by siHSPA7 (FIG. 2C). Immunofluorescence staining showed that the expression of markers of the contractile SMC phenotype, SM22α, and calponin1, was decreased upon oxLDL treatment, and this change was reversed by siHSPA7. The expression of CD68, a marker of macrophage-like cells, was upregulated upon oxLDL treatment, whereas this change was partly inhibited by siHSPA7 (FIG. 2D). The effects of HSPA7 on phenotype markers, particularly CNNI and CD68, were validated using qPCR (FIG. 2E).

### HSPA7 Promotes Inflammatory Changes in HASMCs by Sponging miR-223

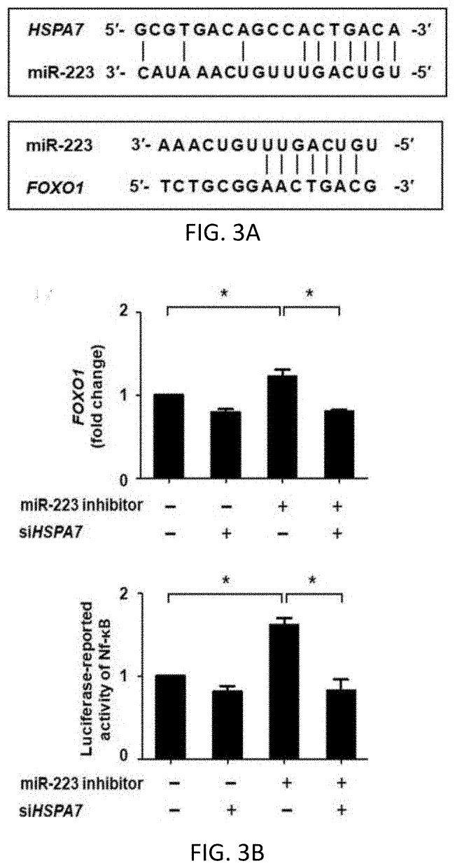

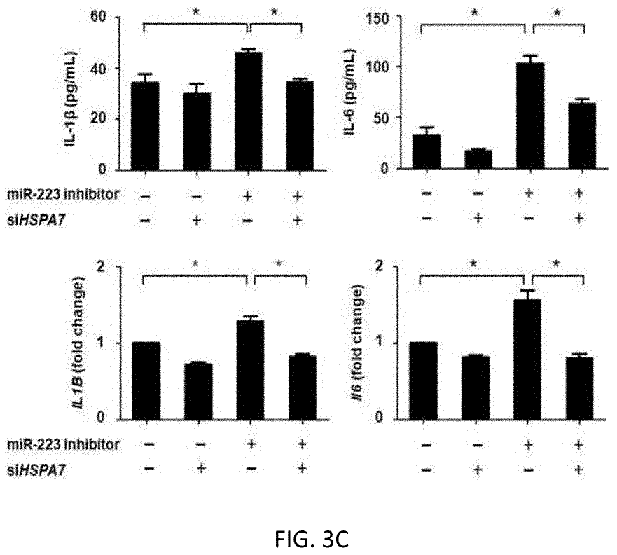

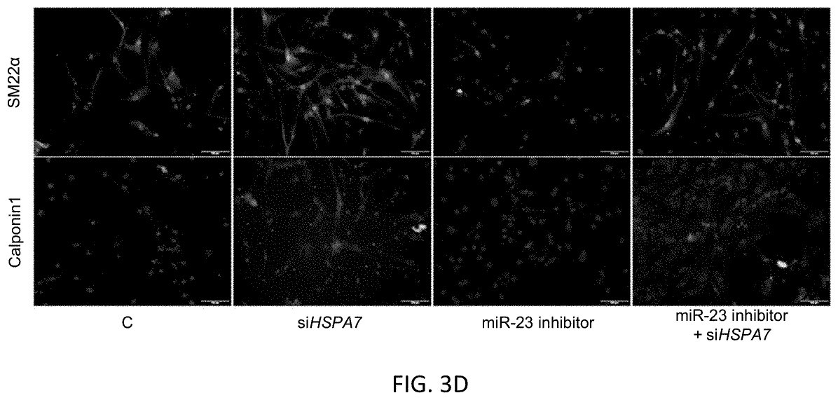

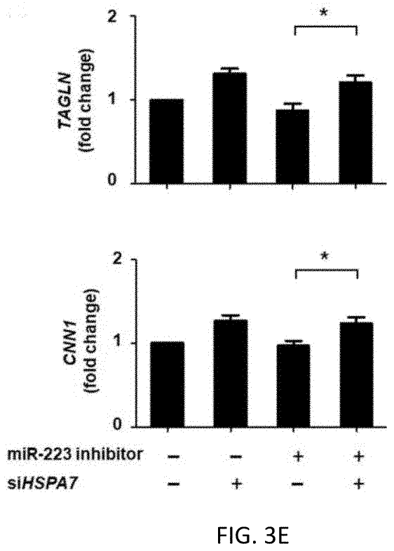

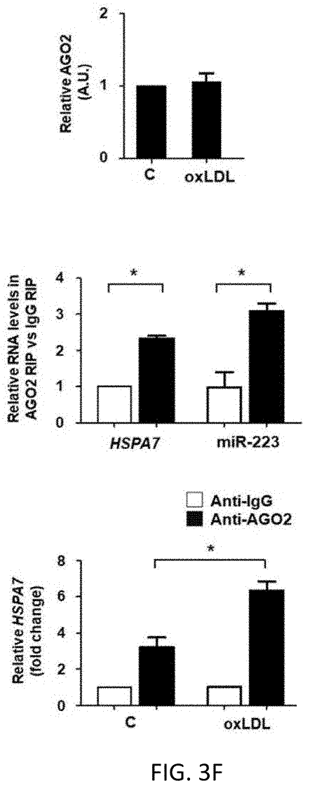

miRcode (http://www.mircode.org/) was used to search for candidate miRNAs interacting with HSPA7, and as a result, it was shown that miR-223 had an optimal site capable of binding to the HSPA7 sequence (FIG. 3A). miRDB (http://mirdb.org/) revealed FOXO1 as a binding target of miR-223 (FIG. 3A). Furthermore, miR-223 has been reported to be associated with cardiac insufficiency and vascular inflammation. HASMCs were transfected with a miR-223 inhibitor and/or siHSPA7 or control siRNA and then treated with 50 mg/mL oxLDL for 24 hours. After 48 hours, luciferase activity was measured. qPCR showed that upregulated expression of FOXO1 by the miR-223 inhibitor was diminished after siHSPA7 treatment. (FIG. 3B) FOXO1 is a transcriptional activator of NF-κB. Although NF-κB activity was elevated due to miR-223 inhibition, this parameter was attenuated upon HSPA7 knockdown (FIG. 3D). 24 hours after the same treatment of HASMCs, the secretion of IL-1β and IL-6 was increased by the miR-223 inhibitor. Increased secretion of chemokines, particularly IL-6, was attenuated after siHSPA7 treatment, which was confirmed again by qPCR (FIG. 3C). In immunofluorescence staining, SM22α and calponin1 expression were not changed upon miR-223 inhibitor treatment, whereas the expression of these markers increased after the addition of siHSPA7 (FIG. 3D). These results were verified using qPCR, particularly for TAGLN gene (FIG. 3E).

### HSPA7 Targets miR-223 in an AGO2-Dependent Manner

miRNA exists in the form of a miRNA ribonucleoprotein complex (miRNP) including AGO2, which is a key component of the RNA-induced silencing complex. To evaluate whether HSPA7 is associated with miRNA, the present inventors conducted an RIP assay in HASMCs using an AGO antibody. AGO2 expression was not different in the cells irrespective of the presence of oxLDL. RIP assays showed that HSPA7 and miR-223 were enriched in the AGO2-containing miRNPs compared to the IgG immunoprecipitates. Furthermore, HSPA7 binding to AGO2 was enhanced in the presence of oxLDL (FIG. 3F).

The features and advantages of the present invention are summarized as follows:

(a) The present invention provides a composition for diagnosing cardio-cerebrovascular disease and a composition for preventing or treating cardio-cerebrovascular disease.

(b) The present invention may provide important clinical information that enables early establishment of treatment strategies by highly reliable prediction of not only the development of cardio-cerebrovascular diseases, including arteriosclerosis, but also the likelihood of future development of cardio-cerebrovascular diseases, based on the expression of lncRNA HSPA7.

(c) In addition, the composition for treating cardio-cerebrovascular disease according to the present invention may inhibit HSPA7 expression, thereby inhibiting the migration of smooth muscle cells and decreasing the expression of inflammatory factors to block the development and progression of atherosclerotic plaques themselves, and thus it may be effectively used as a fundamental therapeutic composition that goes beyond symptomatic therapy such as administration of antithrombotic agents.

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

What is claimed is:

1. A method for predicting or diagnosing cardio-cerebrovascular disease comprising measuring an expression level of lncRNA HSPA7.

2. The method of claim 1, wherein measuring the expression level of lncRNA HSPA7 is carried out using a primer or a probe that binds specifically to the nucleotide sequence of SEQ ID NO: 1.

3. The method of claim 1, wherein the cardio-cerebrovascular disease is selected from the group consisting of myocardial infarction, atherosclerosis, atherothrombosis, coronary artery disease, stable and unstable angina, stroke, vascular stenosis, vascular restenosis, aortic aneurysms, and acute ischemic arteriovascular events.

4. A method for preventing or treating cardio-cerebrovascular disease comprising administering an inhibitor for lncRNA HSPA7 expression a subject in need thereof.

5. The method of claim 4, wherein the inhibitor for lncRNA HSPA7 expression is at least one selected from the group consisting of shRNA, siRNA, miRNA, ribozyme, PNA, and antisense oligonucleotides, which bind specifically to the nucleotide sequence of SEQ ID NO: 1.

6. The method of claim 4, wherein the inhibitor for lncRNA HSPA7 expression inhibits migration of smooth muscle cells.

7. The method of claim 4, wherein the inhibitor for lncRNA HSPA 7 expression reduces secretion of interleukin (IL)-1β and IL-6.

8. The method of claim 4, wherein the cardio-cerebrovascular disease is selected from the group consisting of myocardial infarction, atherosclerosis, atherothrombosis, coronary artery disease, stable and unstable angina, stroke, vascular stenosis, vascular restenosis, aortic aneurysms, and acute ischemic arteriovascular events.

9. A method for screening a composition for preventing or treating cardio-cerebrovascular disease, the method comprising steps of:
(a) bringing a candidate substance into contact with a biological sample containing lncRNA HSPA 7-expressing cells; and
(b) measuring an expression level of lncRNA HSPA 7 in the sample,
wherein the candidate substance is determined as the composition for preventing or treating cardio-cerebrovascular disease, when the expression level of lncRNA HSPA 7 has decreased.

10. The method of claim 9, wherein the lncRNA HSPA7-expressing cells are smooth muscle cells.

11. The method of claim 9, wherein the cardio-cerebrovascular disease is selected from the group consisting of myocardial infarction, atherosclerosis, atherothrombosis, coronary artery disease, stable and unstable angina, stroke, vascular stenosis, vascular restenosis, aortic aneurysms, and acute ischemic arteriovascular events.

12. A composition for predicting or diagnosing cardio-cerebrovascular disease containing, as an active ingredient, an agent for measuring an expression level of lncRNA HSPA7.

13. The composition of claim 12, wherein the agent for measuring the expression level of lncRNA HSPA7 is a primer or a probe that binds specifically to the nucleotide of SEQ ID NO: 1.

14. The composition of claim 12, wherein the cardio-cerebrovascular disease is selected from the group consisting of myocardial infarction, atherosclerosis, atherothrombosis, coronary artery disease, stable and unstable angina, stroke, vascular stenosis, vascular restenosis, aortic aneurysms, and acute ischemic arteriovascular events.

15. A composition for preventing or treating cardio-cerebrovascular disease containing an inhibitor for lncRNA HSPA7 expression as an active ingredient.

16. The composition of claim 15, wherein the inhibitor for lncRNA HSPA7 expression is at least one selected from the group consisting of shRNA, siRNA, miRNA, ribozyme, PNA, and antisense oligonucleotides, which bind specifically to the nucleotide sequence of SEQ ID NO: 1.

17. The composition of claim 15, which inhibits migration of smooth muscle cells.

18. The composition of claim 15, which reduces secretion of interleukin (IL)-1β and IL-6.

19. The composition of claim 15, wherein the cardio-cerebrovascular disease is selected from the group consisting of myocardial infarction, atherosclerosis, atherothrombosis, coronary artery disease, stable and unstable angina, stroke, vascular stenosis, vascular restenosis, aortic aneurysms, and acute ischemic arteriovascular events.


SEQUENCE LISTING (1 sequence)

SEQ ID NO: 1 - DNA, 2493 bp, Homo sapiens
cccgggcgggcgggcgggaggctctcgactgggcgggaaggtgcgggaaggttcgcggcggcggggtcggggaggtgcaaaaggatgaaaagcccgtggaagcggagctgagcagatccgagccgggctggcggcagagaaaccgcagggagagcctcactgctgagcgcccctcgacggcggagcggcagcagcctccgtggcctccagcatccgacaagaagcttcagccatgcaggccccacgggagctcgcggtgggcatcgacctgggcaccacctactcgtgcgtgggcgtgtttcagcagggccgcgtggagatcctggccaacgaccagggcaaccgcaccacgcccagctacgtggccttcaccgacaccgagcggctggtcggggacgcggccaagagccaggcggccctgaacccccacaacaccgtgttcgatgccaagcggctgatcgggcgcaagttcgcggacaccacggtgcagtcggacatgaagcactggcccttccaggtggtgagcgagggcggcaagcccaaggtgcgcgtatgctaccgcggggaggacaagacgttctaccccgaggagatctcgtccatggtgctgagcaagatgaaggagacggccgaggcgtacctgggccagcccgtgaagcacgcagtgatcaccgtgcccacctatttcagtaactcgcagcgccaggccaccaaggacgcgggggccatcgcggggctcaaggtgctgccgatcatcaatgaggccacggcagcagccatcgcctatgggctggaccggcggggcgcgggaaagcgcaacgtgctcatttttgacctgggtgggggcaccttcgatgtgtcggttctctccattgacgccggtgtctttgaggtgaaagccactgctggagatacccacctgggaggagaggacttcgacaaccggctcgtgaaccacttcatggaagaattccggcggaagcatgggaaggacctgagcgggaacaagcgtgccctgcgcaggctgcgcacagcctgtgagcgcgccaagcgcaccccgtcctccagcacccaggccaccctggagatagactccctgttcgagggcgtggacttctacaagtccatcactcgtgcccgctttgaggaactgtgctcagacctcttccgcagcaccctggagccggtggagaaggccctgcgggatgccaagctggacaaggcccagattcatgacttcgtcctggggggagggctccactcgcatccccaaggtgcagaagttgctgcaggacttcttcaacggcaaggagctgaacaagagcatcaaccctgatgaggctgtggcctatgggtctgctgtgcaggcggccgtgttgatgggggacaaatgtgagaaagtgcaggatctcctgctgctggatgtggctcccctgtctctggggctggagacagcaggtggggtgatgaccacgctgatccagaggaacgccactatccccaccaagcagacccagactttcaccacctactcggacaaccagcctggggtcttcatccaggtgtatgaggttgagagggccatgaccaaggacaacaacctgctggggcgttttgaactcattggcatccctcctgccccacatggagtcccccagatagaggtgacgtttgacattgatgctaatggcatcctgagcgtgacagccactgacaggagcacaggtaaggctaacaagatcaccaatgacaagggccggctgagcaaggaggaggtggagaggatggttcatgaagccgagcagtacggggctgaggatgaggcccagagggacagagtggctgccaaaaactcgctggaggcccatgtcttccatgtgaaaggttctttgcaagaggaaagccttagggacaagattcccgaagaggacaggcgcaaagtgcaagacaagtgtcaggaagtccttgcctggctggagcacaaccagctggcagagaaggaggagtatgagcatcagaagagggagctggagcaaatctgtcgccccatcttctccaggctctatggggggcctggtgtccctgggggcagcagttgtagcgctcaagcccaccagggggaccccagcaccggccccatcattgaggaggttgattgaatggcccttcgtgataagtcagctgtgactgtcagggctatgctatgggccttctagactgtcttctatgatcctgcccttcagagatgaagggcttgggggggtcttccctccaaagctagaactttctttccaggataactgaagtcttttgactttttggggggagggcggttcatcctcttctgcttcaaataaaaagtcattaatttattaaaacttgtgtggcactttaacattgctttcacctatattttgtgtattttgttacttgtatgtatgaattttgttatgtaaaatatagttatagacctaaataaacttttaaaactcc